(19) United States
(12) Patent Application Publication
(10) Pub. No.: US 2024/0017017 A1
(43) Pub. Date: Jan. 18, 2024

(54) MEDICAMENT DELIVERY DEVICE

(71) Applicant: SHL MEDICAL AG, ZUG (CH)

(72) Inventor: Anders Boström, Ingarö (SE)

(21) Appl. No.: 18/035,997

(22) PCT Filed: Nov. 15, 2021
(86) PCT No.: PCT/EP2021/081643

§ 371 Date: May 9, 2023

(30) Foreign Application Priority Data
Nov. 27, 2020 (EP) 20210334.7

(51) Int. Cl. A61M 5/32; A61M 5/20

(52) U.S. Cl. CPC A61M 5/3243; A61M 5/20; A61M 5/322

(57) ABSTRACT

A medicament delivery device is presented having a housing, a plunger rod biased in a proximal direction of the medicament delivery device, a plunger holder radially engaging the plunger rod, and wherein the plunger holder has a radial recess, an inner body arranged radially outside the plunger holder and provided with a radially flexible gripping member that engages with the radial recess to hold the plunger holder in a fixed axial position, and a movable sleeve that moves axially in the housing from a first position to a second position, wherein the movable sleeve has a blocking wall which in the first position is arranged radially outside the gripping member and which is prevented from flexing radially outwards by a radially inwards directed force applied onto the blocking wall by the housing, thereby preventing the gripping member to flex radially outwards and disengage from the recess.

### TECHNICAL FIELD

The present disclosure generally relates to medicament delivery devices.

### BACKGROUND

Some medicament delivery devices have an auto-penetration functionality. The delivery member is in this case automatically shifted in the forward direction, penetrating the skin and positioning at a predetermined penetration depth, when medicament is to be administered. Some designs to achieve this use a plunger rod arranged in a plunger holder, with the plunger holder being shifted forward together with the plunger rod during auto-penetration. Before use, the plunger holder should be fixed axially to prevent the auto-penetration functionality to be activated.

An example of a design of the type mentioned above is disclosed in WO2013032389. This document discloses an injection device which has a plunger holder that is held in position by first levers of an inner body. The first levers are held in engagement with the plunger holder by tongues of a sleeve positioned radially outside of the inner body, inside the device housing. The first levers recoil when the sleeve is moved in the distal direction to a position in which openings provided in the sleeve overlap the first levers. The plunger holder is thereby released and moved proximally. The sleeve is moved to the position with overlap by a needle cover that is axially movable inside the injection device.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device.

When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, “circumferentially”, “radial”, “radially”, “rotation”, “rotational” and “rotationally” refer to a direction generally perpendicular to the longitudinal direction and at least partially extending around the longitudinal direction.

As mentioned previously, the plunger holder is biased in the forward direction. This axial biasing force is high to enable a high-force auto-penetration. There is a risk that the tongues of the sleeve could flex radially outwards because the first levers are constantly urged radially outwards by the high biasing force, thereby allowing the first levers to disengage from the plunger holder. The plunger holder could thus potentially accidentally be released, causing auto-penetration.

There is hence provided a medicament delivery device comprising: a housing, a plunger rod biased in a proximal direction of the medicament delivery device, a plunger holder configured to receive the plunger rod and to radially 3o engage with the plunger rod, wherein the plunger holder is configured to be biased in the proximal direction, and wherein the plunger holder has a radial recess, an inner body arranged radially outside the plunger holder and provided with a radially flexible gripping member configured to engage with the radial recess to hold the plunger holder in a fixed axial position, and a movable sleeve configured to be moved axially in the housing from a first position relative to the housing, towards which the movable sleeve is biased, to a second position, wherein the movable sleeve has a blocking wall which in the first position is arranged radially outside the gripping member and which is prevented from flexing radially outwards by a radially inwards directed force applied onto the blocking wall by the housing, thereby preventing the gripping member to flex radially outwards and disengage from the recess, and wherein the movable sleeve has a radial opening arranged proximally relative to the blocking wall, the radial opening being configured to align axially with the gripping member in the second position of the movable sleeve to allow the gripping member to flex radially outwards and release the plunger holder.

Due to the radially inwards directed force applied onto the blocking wall by the housing, the blocking wall is unable to flex radially outwards to a degree that would allow the gripping member to disengage from the plunger holder. The risk of accidental disengagement of the gripping member from the plunger holder can thereby be eliminated.

The radially inwards directed force may be transmitted from the housing by direct contact between the blocking wall and the housing or indirectly by an intermediate component arranged between the blocking wall and the housing. The intermediate component may for example be an outer sleeve of the inner body.

According to one embodiment the medicament delivery device is an injector, such as an auto-injector, e.g. an auto-injector with auto-penetration functionality.

According to one embodiment the blocking wall extends between the gripping member and an inner surface of the housing or a radially outwards arranged surface of the inner body to restrict radial flexing of the blocking wall.

The radially outwards arranged surface is oriented to face the central axis of the medicament delivery device. The radially outwards arranged surface is a radially inwards facing surface.

The radially outwards arranged surface may form part of an outer sleeve of the inner body. The outer sleeve may be in contact with the inner surface of the housing. In this way, a radial force acting on the outer sleeve may be transmitted to the blocking wall.

According to one embodiment the blocking wall extends all the way to the inner surface of the housing or to the radially outwards arranged surface. The blocking wall is thereby unable to move radially.

The blocking wall may thus have a thickness corresponding to the distance between the gripping member and the inner surface of the housing or the radially outwards arranged surface of the inner body when the gripping member engages with the plunger holder.

The blocking walls may bear against the inner surface of the housing or the radially outwards arranged surface of the inner body. Friction can thereby be created between the blocking wall and the inner surface, which could keep the movable sleeve axially fixed or immovable in case the medicament delivery device would accidentally be dropped. The risk of accidental activation of the medicament delivery device is thereby reduced.

According to one embodiment the blocking wall is provided with a plurality of ribs, wherein the ribs extend between the gripping member and the inner surface of the housing or the radially outwards arranged surface.

The ribs fill up the radial space between the movable sleeve and the inner surface of the housing or the radially outwards arranged surface.

By using ribs, the friction can be somewhat reduced compared to solid blocking walls, thereby facilitating activation of the medicament delivery device by moving the movable sleeve towards the second position. Moreover, less material is required for making the movable sleeve, reducing the environmental footprint and production costs.

According to one embodiment the ribs are axially extending. The ribs may extend parallel with each other and with the longitudinal axis of the medicament delivery device.

One embodiment comprises a filling sleeve arranged radially outside of the movable sleeve and the gripping member, wherein the filling sleeve extends between an outer surface of the blocking wall and an inner surface of the housing or a radially outwards arranged surface of the inner body to prevent radial flexing of the blocking walls.

According to one embodiment the filling sleeve is provided with a plurality of outer sleeve ribs, wherein the outer sleeve ribs extend all the way to the inner surface of the housing or the radially outwards arranged surface.

According to one embodiment the filling sleeve has an axial through-opening configured to receive a distal end portion of the movable sleeve when the movable sleeve is moved towards the second position.

According to one embodiment the filling sleeve has a side wall arranged radially outside the blocking wall when the movable sleeve is in the first position, wherein the side wall is provided with a filling sleeve opening arranged proximally relative to a surface of the side wall that prevents the blocking wall to flex radially outwards in the first position of the movable sleeve, wherein the radial opening is configured to axially align with the filling sleeve opening in the second position of the movable sleeve.

Generally, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 shows a perspective view of an example of a medicament delivery device;

FIG. 2 is an exploded view of the medicament delivery device in FIG. 1;

FIG. 3 shows some internal components of the medicament delivery device in

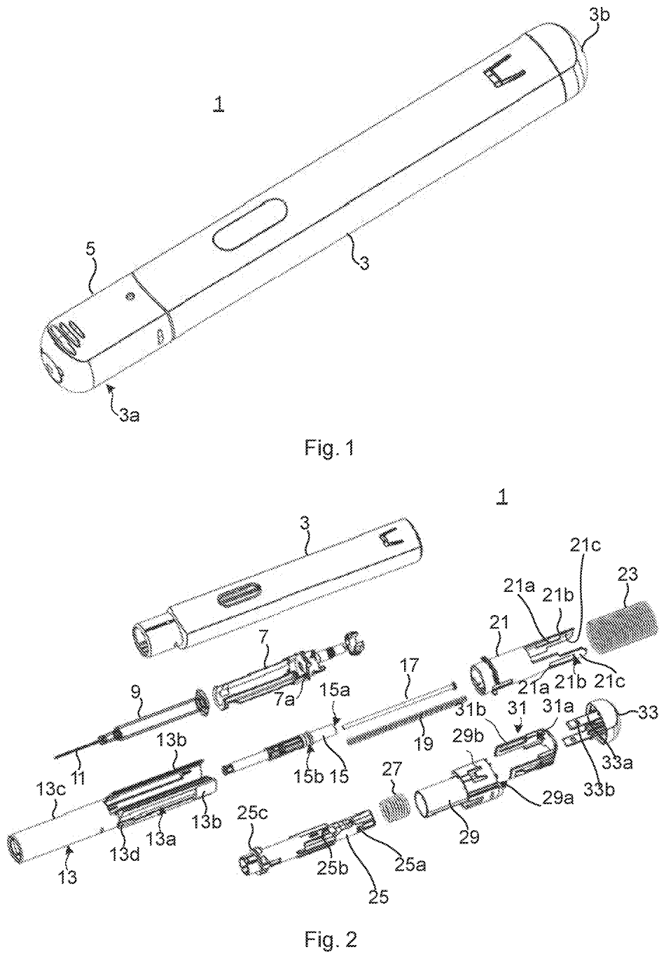

FIG. 1;

FIGS. 4A-4C show a distal portion of the medicament delivery device in FIG. 1 in a longitudinal section during different stages of operation;

FIG. 5 shows a distal portion of a movable sleeve of another example of a medicament delivery device; and

FIGS. 6A-6B show longitudinal sections of a proximal end portion of an example of a medicament delivery device employing the movable sleeve shown in FIG. 5.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like elements throughout the description.

FIG. 1 shows a perspective view of an example of a medicament delivery device 1. The medicament delivery device 1 may for example be an injector such as an auto-injector. The medicament delivery device 1 may be an injector with auto-penetration functionality.

The medicament delivery device 1 comprises a housing 3. The medicament delivery device 1 has a proximal end 3a, which is a front end, and a distal end 3b, which is a rear end.

The medicament delivery device 1 has a removable front cap 5 which when attached to the housing 3 forms the proximal end 3a.

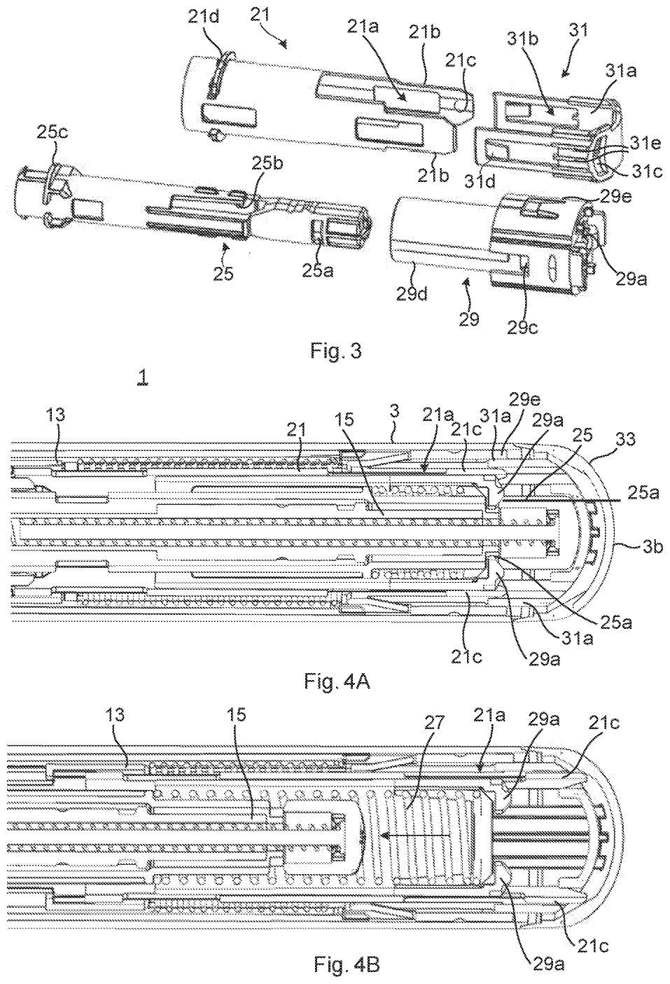

FIG. 2 shows an exploded view of the medicament delivery device 1, without the front cap 5.

The medicament delivery device 1 comprises a medicament container holder 7 configured to be slidably arranged in the housing 3. The medicament container holder 7 has radially outwards extending protrusions 7a.

The medicament delivery device 1 may also comprise a medicament container 9 filled with medicament and provided with a delivery member ii, such as a needle. The medicament container holder 7 is configured to receive the medicament container 9.

The medicament delivery device 1 comprises a delivery member cover 13. The delivery member cover 13 is configured to be slidably arranged in the housing 3. The delivery member cover 13 is configured to be moved axially in the housing 3 between an extended position relative to the housing 3 and a retracted position.

The exemplified delivery member cover 13 has slots 13a extending axially along a distal portion of the delivery member cover 13. The delivery member cover 13 may have a cylindrical or essentially cylindrical proximal portion 13c, and two legs 13b extending distally from the cylindrical proximal portion 13c. The legs 13b extend parallel with each other. The slots 13a may be arranged in a respective leg 13b.

The delivery member cover 13 may have flexible tabs 13d arranged aligned with and proximally relative to a respective slot 13b. The tabs 13d may be inclined, gradually extending radially outwards in the distal direction. The tabs 13d may extend over a proximal portion of a respective one of the slots 13a.

The slots 13a are configured to receive a respective one of the protrusions 7a of the medicament container holder 7. The protrusions 7a are configured to run in the slots 13a during auto-penetration, as will be explained in more detail in the following.

The medicament delivery device 1 comprises a hollow plunger rod 15 having a distal opening 15a, a rod 17 configured to be arranged in the hollow plunger rod 15, and a first resilient member 19 arranged around the rod 17 and configured to bias the plunger rod 15 in the proximal direction. The plunger rod 15 has a circumferentially extending groove 15b. The first resilient member 19 may for example be a spring such as a coil spring.

The medicament delivery device 1 comprises a movable sleeve 21. The movable sleeve 21 is configured to be moved axially inside the housing 3 between a first position and a second position. The first position is a default position of the movable sleeve 21. The second position is more distal relative to the first position.

The movable sleeve 21 has distally extending sleeve legs 21b provided with a respective radial opening 21a.

The delivery member cover 13 is configured to be arranged proximally relative to the movable sleeve 21 with a distal surface of the delivery member cover 13 bearing against as proximal surface of the movable sleeve 21. The delivery member cover 13 is configured to move the movable sleeve 21 from the first position to the second position when the delivery member cover 13 is moved from the extended position to the retracted position.

According to one variation, the delivery member cover and the movable sleeve could be integrated forming a single component.

The medicament delivery device 1 comprises a second resilient member 23. The second resilient member 23 is configured to bias the movable sleeve 21 towards the first position. The second resilient member 23 is thus configured to bias the delivery member cover 13 towards the extended position. The second resilient member 23 may for example be a spring such as a coil spring.

The medicament delivery device 1 comprises a plunger holder 25. The plunger holder 25 is elongated and hollow and configured to receive the plunger rod 15.

The plunger holder 25 has radial recesses 25a. The radial recesses 25a are provided in the outer surface of the plunger holder 25. The radial recesses may be arranged in a distal portion of the plunger holder 25. With distal portion is in this case meant the distal half of the plunger holder 25.

The plunger holder 25 has radially flexible arms 25b which can be urged towards the hollow interior of the plunger holder 25. The flexible arms 25b are configured to engage with the circumferentially extending groove 15b of the plunger rod 15. The plunger holder 25 thereby holds the plunger rod 15 in an axially fixed position.

The plunger holder 25 has a radial flange 25c configured to engage with or bear against the medicament container holder 7.

The medicament delivery device 1 comprises a third resilient member 27 configured to bias the plunger holder 25 in the proximal direction. The third resilient member 27 may for example be a spring such as a coil spring.

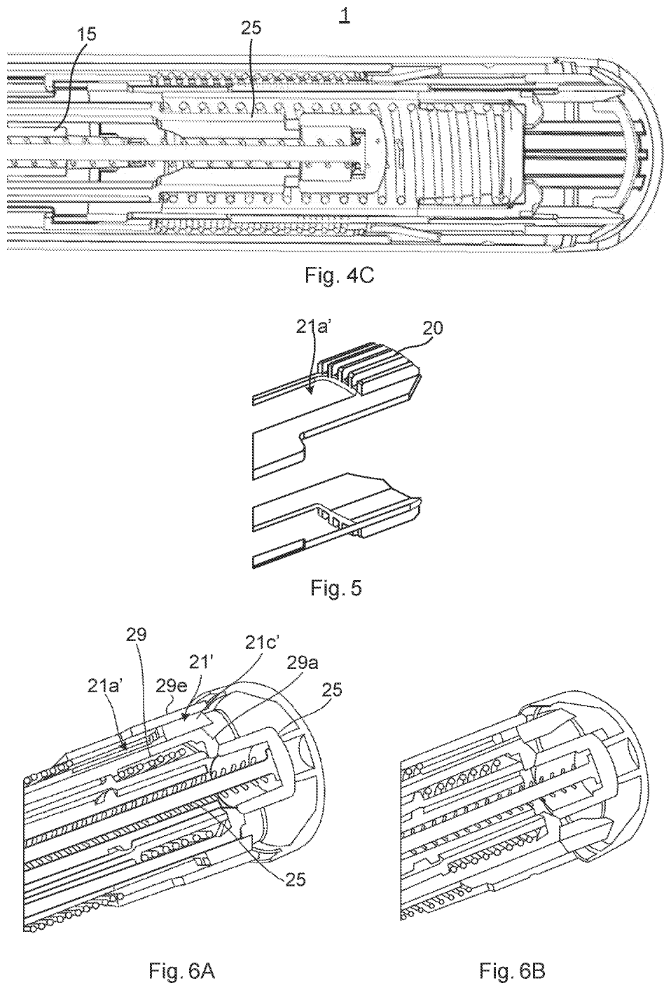

The medicament delivery device 1 comprises an inner body 29 configured to be arranged radially outside the plunger holder 25. The inner body 29 comprises radially flexible gripping members 29a, better seen in FIG. 3, configured to engage with a respective radial recess 25a of the plunger holder 25. The plunger holder is thereby held fixed in an axial position.

The sleeve legs 21b form blocking walls tic configured to bear against the gripping members 29a, to urge the gripping members 29a radially inwards for engagement with the radial recesses 25a when the movable sleeve 21 is in the first position. When the delivery member cover 13 is moved from the extended position to the retracted position, the movable sleeve 21 is moved to the second position. This causes the radial openings Zia to align with the gripping members 29a. The gripping members 29a are thereby allowed to flex radially outwards to disengage from the plunger holder 25. The plunger holder 25 is thereby released and moved in the proximal direction due to the biasing force from the second resilient member 27.

The exemplified medicament delivery device 1 comprises a filling sleeve 31 arranged radially outside of the movable sleeve 21 and the gripping members 29a. In the present example, the inner body 29 has an inner and an outer sleeve arranged concentrically. The filling sleeve 31 is arranged in the radial space between the inner and the outer sleeve.

The filling sleeve 31 has side walls Sia arranged to bear against the blocking walls 21c of the movable sleeve 21 when the movable sleeve 21 is in the first position. The side walls 31 are prevented from radial movement by the outer sleeve of the inner body 29 and by the inner surface of the housing 3 which bears against the outer sleeve. A radially inwards directed force applied by the housing 3 onto the outer sleeve is transmitted to the blocking walls tic via the side walls 31a. The side walls 31 thereby prevent radial outward flexing of the blocking walls 21c. As previously explained, the blocking walls 21 are subjected to radially outwards directed forces from the gripping members 29a because of the proximally directed force applied to the plunger holder 25 by the third resilient member 27.

The filling sleeve 31 has radial filling sleeve openings 31b arranged in the side walls 31. The filling sleeve openings 31b are arranged proximally relative to the surfaces of the side walls 31 that prevent the blocking walls 21c to flex radially outwards in the first position of the movable sleeve 21. The filling sleeve openings 31b are arranged radially outside the blocking walls tic in the first position of the movable sleeve 21. A distal end portion of each sleeve leg 21b and blocking wall 21c is arranged distally relative to the radial openings 21a and radially bears against a respective side wall 31a. In a preferred embodiment, the filling sleeve may comprise multiple ribs, longitudinally extending along the side wall 21a from the distal end to the proximal end; those ribs are configured to be a axial stop of the filling sleeve, so that the axial position of the filling sleeve openings can be fixed.

The medicament delivery device 1 comprises a rear cap 33 configured to be connected to the inner body 29. The rear cap 33 has proximally extending rear cap arms 33a provided with openings 33b configured to engage with radial protrusions 29b of the inner body 29.

FIG. 3 shows close-up views of the movable sleeve 21, the filling sleeve 31, the plunger holder 25 and the inner body 20.

The movable sleeve 21 has a flange 21d configured to bear against a distal end of the delivery member cover 13. In a preferred example, the flange 21d also configured to be a seat for the second resilient member 23, so that the second resilient member is configured to bear against the flange 21d. The delivery member cover 13 can thereby move the movable sleeve 21 from the first position to the second position when the delivery member cover 13 is moved from the extended position towards the retracted position.

The filling sleeve 31 has blocking members 31d, preferably, the blocking members 31d is flexible. The blocking member 31d is configured to bear against proximal inner body surfaces 29c to prevent the filling sleeve 31 from moving axially in the distal direction. The blocking members 31d may be radially flexible inclined tabs. This facilitates assembly of the medicament delivery device 1, as the filling sleeve 31 may be attached to the inner body 31 by sliding it in between the inner and outer sleeves 29d and 29e of the inner body 29 from the distal end of the inner body 29. In a preferred example, the filling sleeve 31 is axially secured between the blocking members 31d and the 3o proximal inner body surfaces 29c.

The filling sleeve could alternatively be configured to move in the distal direction concurrently with the movable sleeve when the movable sleeve is moved from the first position towards the second position. The blocking member could in this case for example be pyramid-shaped to enable movement also in the distal direction when a force over a threshold value acts on the movable sleeve in the distal direction.

The filling sleeve 31 has a distal end provided with axial through-openings 31c configured to receive the sleeve legs 21b of the movable sleeve 21 when the movable sleeve 21 is moved axially from the first position towards the second position. The blocking walls 21c may thereby move distally from a position in which they are axially aligned with the side walls 31a distally past the side walls 31a at least to such a degree that the radial openings 21a become axially and circumferentially aligned with the filling sleeve openings 31b when the movable sleeve 21 has been moved to the second position. The gripping members 29a are thereby able to flex radially outwards, releasing the plunger holder 25.

The radially flexible arms 25b are arranged inside the inner sleeve 29d when the movable sleeve 21 is in the first position and the plunger holder 25 is engaged with the inner body 29. The radially flexible arms 25b are thereby prevented from disengagement from the plunger rod 15.

The filling sleeve 31 may have a plurality of outer sleeve ribs 31e extending in the axial direction parallel with each other on an outer surface of the side walls 31a. The outer sleeve ribs 31e extend all the way to a radially outwards arranged surface of the inner body 29, in this example the inner surface of the outer sleeve 29e.

The operation of the exemplified medicament delivery device 1 will now be shown with reference to FIGS. 4A-4C.

FIG. 4 shows a distal portion, or rear portion, of the medicament delivery device 1 in a default state before the medicament delivery device 1 has been activated for medicament administration.

The delivery member cover 13 is in the extended position, and the movable sleeve 21 is in the first position. The gripping members 29a are arranged in a respective recess 25a and the plunger holder 25 is thus axially fixed. The blocking walls 21c of the movable sleeve 21 bear against the gripping members 29a. The side walls 21a of the filling sleeve 31 are arranged radially outside the blocking walls 21c. The side walls 31a extend radially to the inner surface of the outer sleeve 29e of the outer body 29. . The gripping members 29a are thereby prevented to disengage from the plunger holder 25.

FIG. 4b shows a situation where the plunger holder 25 has been released from the inner body 29 and auto-penetration has been performed. The delivery member cover 13 has in this case been pushed inwards into the housing 3 from the extended position to the retracted position. This is typically done by the user pressing the medicament delivery device 1 towards the site of injection. The movement of the delivery member cover 13 has moved the movable sleeve 21 in the distal direction from the first position to the second position. The sleeve legs 21b of the movable sleeve 21 have moved through the axial through-openings 31c of the filling sleeve 31. The radial openings 21a have become axially aligned with the filling sleeve openings 31b and the gripping members 29a. The gripping members 29a have thereby been able to flex radially outwards, and the plunger holder 25 has been released and moved in the proximal direction together with the plunger rod 15 due to the biasing in the proximal direction by the third resilient member 27. The plunger holder 25 pushes the medicament container holder 7 in the proximal direction as the plunger holder 25 is moved proximally by the force of the third resilient member 27. Auto-penetration functionality is thereby provided.

As the medicament container holder 7 is moved in the proximal direction, the protrusions 7a slide along the slots 13a until they reach the tabs 13d which force the medicament container holder 7 to decelerate before it stops. This marks the end of the auto-penetration phase of the medicament administration.

FIG. 4c shows the medicament delivery device 1 after medicament expulsion. When the medicament container holder 7 has reached the proximal end of the slots 13a the plunger holder 25 has moved so far in the proximal direction that the radially flexible arms 25b have moved out from the inner sleeve 29d, thereby enabling the radially flexible arms 25b to flex radially outwards. The plunger rod 15 is as a result released from engagement with the plunger holder 25. The plunger rod 15 is thus moved further into the medicament container 9 for medicament expulsion from the medicament container 9.

Another example of a medicament delivery device will now be described with reference to FIGS. 5-6B.

The medicament delivery device 1′, shown in FIG. 6A is similar to the medicament delivery device 1. The medicament delivery device 1′ however does not have a filling sleeve. Instead, the medicament delivery device 1′ has a movable sleeve 21′ with thicker blocking walls 21c' extending all the way to the inner surface of the outer sleeve 29e of the inner body 29. In other aspects, the movable sleeve 21′ has the same design as the movable sleeve 21 in the previous example.

A distal end portion of the movable sleeve 21′ is shown in more detail in FIG. 5. The blocking wall 21c′ is provided with a plurality of axially extending ribs 20 configured to extend to the inner surface of the outer sleeve 29e.

FIG. 6c depicts the situation when the movable sleeve 21′ has been moved to the second position for releasing the plunger holder 25.

As one alternative to the above examples, it is envisaged that the inner body could be arranged without the outer sleeve. In this case, the thicker blocking walls would extend all the way to the inner surface of the housing, or the side walls of the filling sleeve would extend all the way to the inner surface of the housing.

The inventive concept has mainly been described above with reference to a few examples. However, as is readily appreciated by a person skilled in the art, other embodiments than the ones disclosed above are equally possible within the scope of the inventive concept, as defined by the appended claims.

1-9. (canceled).

10. A medicament delivery device comprising:
a housing,
a plunger rod biased in a proximal direction of the medicament delivery device,
a plunger holder configured to receive the plunger rod and to radially engage with the plunger rod, wherein the plunger holder is configured to be biased in the proximal direction, and wherein the plunger holder has a radial recess,
an inner body arranged radially outside the plunger holder and provided with a radially flexible gripping member configured to engage with the radial recess to hold the plunger holder in a fixed axial position, and
a movable sleeve configured to be moved axially in the housing from a first position relative to the housing, towards which the movable sleeve is biased, to a second position,
wherein the movable sleeve has a blocking wall which in the first position is arranged radially outside the gripping member and which is prevented from flexing radially outwards by a radially inwards directed force applied onto the blocking wall by the housing, thereby preventing the gripping member to flex radially outwards and disengage from the recess, and wherein the movable sleeve has a radial opening arranged proximally relative to the blocking wall, the radial opening being configured to align axially with the gripping member in the second position of the movable sleeve to allow the gripping member to flex radially outwards and release the plunger holder.

11. The medicament delivery device as claimed in claim 10, wherein the blocking wall extends between the gripping member and an inner surface of the housing or a radially outwards arranged surface of the inner body to restrict radial flexing of the blocking wall.

12. The medicament delivery device as claimed in claim 11, wherein the blocking wall extends all the way to the inner surface of the housing or to the radially outwards arranged surface.

13. The medicament delivery device as claimed in claim 11, wherein the blocking wall is provided with a plurality of ribs, wherein the ribs extend between the gripping member and the inner surface of the housing or the radially outwards arranged surface.

14. The medicament delivery device as claimed in claim 13, wherein the ribs are axially extending.

15. The medicament delivery device as claimed in claim 10, comprising a filling sleeve arranged radially outside of the movable sleeve and the gripping member, wherein the filling sleeve extends between an outer surface of the blocking wall and an inner surface of the housing or a radially outwards arranged surface of the inner body to prevent radial flexing of the blocking wall.

16. The medicament delivery device as claimed in claim 15, wherein the filling sleeve is provided with a plurality of outer sleeve ribs, wherein the outer sleeve ribs extend all the way to the inner surface of the housing or the radially outwards arranged surface.

17. The medicament delivery device as claimed in claim 15, wherein the filling sleeve has an axial through-opening configured to receive a distal end portion of the movable sleeve when the movable sleeve is moved towards the second position.

18. The medicament delivery device as claimed in claim 15, wherein the filling sleeve has a side wall arranged radially outside the blocking wall when the movable sleeve is in the first position, wherein the side wall is provided with a filling sleeve opening arranged proximally relative to a surface of the side wall that prevents the blocking wall to flex radially outwards in the first position of the movable sleeve, wherein the radial opening is configured to axially align with the filling sleeve opening in the second position of the movable sleeve.

19. A medicament delivery device comprising:
a housing;
a plunger rod;
a plunger holder comprising a radial recess, where the plunger holder radially engages the plunger rod and where plunger rod holder is biased in the proximal direction;
an inner body surrounding the plunger holder and comprising a radially flexible gripping member that engages the radial recess holding the plunger holder in a fixed axial position; and
a sleeve biased and movable axially relative to the housing from a first position to a second position,
wherein the sleeve has a blocking wall which in the first position is positioned adjacent the gripping member preventing the gripping member from flexing radially outward and disengaging from the recess, and
wherein the sleeve has a radial opening arranged proximally relative to the blocking wall, where the radial opening aligns axially with the gripping member when the sleeve is in the second position such that the gripping member flexes radially outwards releasing the plunger holder to move axially.

20. The medicament delivery device of claim 19, wherein the blocking wall exerts a radially inward directed force caused by engagement with an inside surface of the housing.

21. The medicament delivery device of claim 19, wherein the blocking wall comprises a plurality of ribs that extend between the gripping member and the inner surface of the housing.

22. The medicament delivery device of claim 19 further comprising a filling sleeve positioned outside of the movable sleeve and the gripping member and extends between an outer surface of the blocking wall and an inner surface of the housing.
